(19) United States
(12) Patent Application Publication
(10) Pub. No.: US 2012/0093932 A1
(43) Pub. Date: Apr. 19, 2012

(54) TARGETED SUSTAINED-RELEASE MICROSPHERE OF VASCULAR OCCLUSIVE AGENT CONTAINING SODIUM ALGINATE AND SORAFENIB, PRODUCTION METHOD AND USE THEREOF

(75) Inventors: Xinjian Li, Beijing (CN); Hong Hong, Beijing (CN); Ge Lu, Beijing (CN)

(73) Assignee: Beijing Shengyiyao Science & Technology Development Co., Ltd., Beijing (CN)

(21) Appl. No.: 13/264,709

(22) PCT Filed: Apr. 14, 2010
(86) PCT No.: PCT/CN10/71749

§ 371 Date: Dec. 9, 2011

(30) Foreign Application Priority Data
Apr. 16, 2009 (CN) 200910082449.6

(51) Int. Cl. A61K 9/14; A61P 35/00; A61K 31/44

(52) U.S. Cl. 424/489; 514/350

(57) ABSTRACT

A targeted sustained-release microsphere vascular embolizing agent, the production method and the use thereof are disclosed. The microsphere comprises sodium alginate as the carrier and sorafenib as the targeted anti-tumor medicine and sorafenib is encapsulated by sodium alginate. The weight ratio of sorafenib to sodium alginate is 1:1˜1:30. The microspheres are used for manufacturing medicament for the treatment of solid tumors with advantages including high medicine concentration in the target regions with reduced systemic dosage and toxic and side effects.

### TECHNICAL FIELD

The present invention relates to a microsphere vascular embolizing agent comprising anti-tumor drug, the preparation method and the use thereof. The present invention especially relates to a targeted sustained-release sodium alginate microsphere vascular embolizing agent containing sorafenib, the preparation method and the use thereof.

### BACKGROUND TECHNOLOGIES

Sorafenib is a novel diaryl urea, under the chemical name of 4-{4-[3-(4-chloro-3-trifluoro-phenyl)-ureido]-phenoxyl}-pyridine-2-carboxylic methylamine, whose molecular weight is 464.8 g/mol. The sorafenib used in clinic is its tosylate salt. The molecular formula of sorafenib tosylate is C₂₁H₁₆C₁F₃N₄O₃.C₇H₈O₃S, the molecular weight is 637.0 g/mol and the chemical formula is shown below:

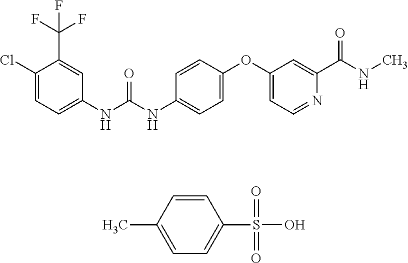

The melting point of sorafenib tosylate is 225-235° C., and is a kind of tasteless solid, intermediate between white and brown. It is heat-stable and nonabsorbent; its solubility is low in aqueous solution and becomes higher a little bit under the strongly acid condition. It is slightly soluble in alcohol but can be dissolved in polyethylene glycerol 400. Sorafenib is a multi-target tumor-targeting therapeutic drug, which was co-developed by Bayer and ONYX during their collaboration since 1994. It is the first tumor-targeting drug approved by the U.S. Food and Drug Administration (FDA), for the treatment of metastatic renal cancer. Sorafenib was officially approved by U.S. FDA in December 2005 for use in the treatment of advanced renal cancer and it therefore became the first commercially available oral multikinase inhibitor. It was authorized to be marketed in China in End November of 2006.

Sorafenib acts as tyrosine kinase inhibitor, angiogenesis inhibitor, as well as vascular endothelial growth inhibitor. The survival, growth and metastasis of tumor are dependent on the effective cell proliferation and angiogenesis of tumor. Ras (GTP binding protein)/Raf signal transduction is a key pathway involved in tumor cell proliferation and angiogenesis. Raf is a serine/threonine (Ser/Thr) protein kinase and is the downstream effector enzyme of Ras protein. Once Raf is activated, mitogen-activated protein kinase (MEK) 1 and 2 are both triggered, which in turn cause extracellular signal-regulated kinase (ERK) 1 and 2 to be activated by phosphorylation and then translocated to nucleus. Transcription initiation and translation activation pathways are therefore triggered, resulting in cell proliferation. So this signal transduction pathway plays a direct role in regulating tumorigenesis and tumor developement in various human tumor tissues. On one hand, sorafenib inhibits RAS/RAF/MEK/ERK signal transduction pathway by inhibiting the activity of RAF so as to inhibit tumor cell growth directly. On the other hand, sorafenib interrupts neovascularization of tumor and also cuts off the nutrition supply of tumor cells by inhibiting the activity of several tyrosine kinase receptors involved in neovascularization and development of tumor, including vascular endothelial growth factor receptor 2 (VEGFR-2), REGFR-3, platelet derived growth factor receptor β (PDGFR-β) and proto-oncogene C-kit, so as to inhibit the growth of tumor cells indirectly. Thus, sorafenib has a dual anti-tumor function.

Sorafenib is mainly metabolized in liver through CYP3A4 enzyme oxidative metabolism and UGTIA9 glucuronic acid metabolism. Eight of metabolic products have been identified and five of them have been measured in blood. The predominant metabolic product pyridine N-Oxide, accounting for 9%˜16% of all metabolic products, plays a similar role to sorafenib in vitro. When orally taking a single dose of a solution containing 100 mg sorafenib, 77% of sorafenib is excreted in the faeces within 14 days, where 15% is excreted unchanged; while 19% of sorafenib is excreted as glucuronic acid metabolic products in urine. Sorafenib is the first drug allowed to enter clinical trials among congeneric compounds. The preliminary results of the clinical studies suggest that sorafenib has anti-tumor effects on solid tumors such as renal cancer, liver cancer, melanoma, non-small cell lung cancer, gastric cancer, ovarian cancer and the like.

The common side effects of sorafenib include skin redness, rash, itching, hair loss or patchy hair loss, frequent diarrhea and/or enterokinesia relaxation, nausea or vomiting, oral ulcer, fatigue, appetite loss (decrease), high blood pressure, hand-foot syndrome and the like. Among the common side effects of sorafenib, dermal toxicity and gastrointestinal responses are the main reasons for decreasing dosage or halting medication. During the treatment with oral administration of sorafenib, the most common side effects are the ones caused in gastrointestinal track, wherein 95% is gastrointestinal response, 58% is diarrhea, 30% is nausea, 24% is vomiting, and 47% is indigestion accompanied by appetite loss.

The potential side effects announced by the U.S. FDA include birth defect or death of fetus. Thus, any methods of birth control should be adopted for both male and female during the treatment and for two weeks after stopping taking sorafenib. The potential side effects such as redness, pain, swelling or blistering in palm and thenar may be observed as well. Blood pressure should be checked every week during the first six weeks of the treatment. If high blood pressure is caused while taking the medication, it should be treated in time. If patient has any potential heart problems, doctors should be informed before medication, since some side effects in heart may be caused during the treatment.

A number of genes and kinases are involved in tumorigenesis and tumor development, so targeted therapy has become one of the hottest research fields nowadays. Based on the further disclosure of the molecular biology mechanism of tumorigenesis, sorafenib was developed successfully as a novel drug possessing a unique multi-target anti-tumor activity. Its successful application in clinic inaugurated a new era of biological targeted therapy on tumors. In the aspects of the mechanism of action as well as the clinical trial results, sorafenib is different from chemotherapeutic drugs as it works mainly by inhibiting growth of tumor cells instead of via cytotoxic effects. The clinical trial results have verified that the second-line treatment of advanced renal cell carcinoma with sorafenib can prolong PFS, OS and TTP markedly. There are no doubts that the biggest concerns at the clinical trial stage nowadays are how to further enhance therapeutic effects of sorafenib and how to seek available markers to predict its therapeutic effects. Sorafenib is a novel multi-kinase inhibitor, which can inhibit not only RAF-MEK-ERK pathway but also tyrosine kinase receptors so as to result in the inhibition of tumor growth and angiogenesis. The Phase I clinical trial showed that it is tolerated and effective to take 400 mg orally twice a day and the most common side effects caused are diarrhea and skin lesion. The Phase II clinical trial suggested that sorafenib has anti-tumor activities on liver cancer and renal cancer, respectively. The Phase III clinical trial on advanced renal cancer has proved that the tumor in most of patients was shrunk markedly and the median survival time was prolonged dramatically too. Currently, a number of Phase III clinical trials, such as the Phase III clinical trial of treating liver cancer with sorafenib in China, are still underway. Thus, it is believed that more inspiring outcomes will come up and new hope of treating tumors may also arise. While holding up hopes, it should be noted that a great many problems are pending to be resolved, for example, the fact that it is difficult for drugs to penetrate tumor tissues during medication. Further clinical trials should be performed to overcome the difficulty for drugs of reaching tumor tissues and resolve the problem of treating with low concentration.

A Phase II study carried out by Ghassan et al. on the treatment of liver cancer with sorafenib has shown that the monotherapy with sorafenib has some therapeutic effects on liver cancer. Although the researchers deem that the monotherapy with sorafenib is not very effective, the effect of sorafenib is close to that of combined chemotherapy. The mechanism of action as well as the lower toxicity of sorafenib also allows it to be combined with other anti-cancer drugs to further enhance therapeutic effects. This study is a multi-center Phase III clinical trial in the Asian-Pacific area focusing on middle or advanced liver cancer cases where the patients are unable or unwilling to undergo surgery. As the nosogenesis of liver cancer is being disclosed and new molecular targeted drugs are being studied, patients suffering from advanced liver cancer may be offered an opportunity to receive targeted therapy. Most of liver cancer is descended from hepatocirrhosis so the patients with liver dysfunction and in poor physical condition benefit very little from chemotherapy and radiotherapy. Rapid advances in molecular targeted drugs provide alternative methods to treat liver cancer. In a word, molecular targeted therapy with good targets and low toxicity shows a broad prospect of treating advanced liver cancer. Thus, this kind of drug is probably one of the most potential and promising methods to treat liver cancer in the future.

Primary carcinoma of liver is an extremely malignant tumor. Although excision may be the first therapy, around 70% of patients are already in the middle or terminal stage while diagnosed. At that time, broad lesions or even metastasis has is already happened, which is usually accompanied by hepatocirrhosis, so that the tumor cannot be excised surgically in those cases. Transcatheter arterial chemoembolization (TACE) is one of the key therapies to treat middle and advanced liver cancer and it works by utilizing chemotherapeutic agents as well as embolism. Since 90%-95% of the blood supply of liver cancer is from hepatic artery, infusion of chemotherapeutic agents and embolism through hepatic artery may cause ischemic necrosis of tumors by blocking their blood supply, which allows high-dose agents work particularly on tumors for a longer time so as to improve therapeutic effects eventually. Additionally, treating with TACE before liver cancer surgery may lead to necrosis, absorption and fibrosis of tumor tissues and formation of thick fibrous capsule, all of which will reduce the amount of bleeding in the operation and prevent tumor cells from spreading that may be caused by surgical procedures or extrusion. Furthermore, the activities and toxicities of some drugs will be decreased in liver, which can hardly be reached by intravenous injections.

Tumors are one of world's deadliest diseases nowadays. The clinical treatments such as surgery, radiotherapy, chemotherapy and the like are effective methods to remove tumor mass. However, surgical resection can only be applied to visible tumors but have no effects on invisible subclinical focuses, tumor cells spreading to the surrounding normal tissues through the lymphatic system or bloodstream, or tumor cells infiltrating the surroundings directly. Radiotherapy is a treatment via local radiation so it is unable to kill the tumors outside the radiation area and has no effects on those insensitive tumor cells either. Chemotherapy is a systematic treatment which has poor selective inhibition effects on tumor cells and even has no effects on dormant tumor cells. For those reasons, several new methods and techniques have been developed to treat tumors in the recent years. Among them, molecular targeted therapy turns out to be a hotspot and even a trend of current researches. On the basis of tumor molecular biology, the molecular targeted therapy works by utilizing specific agents or drugs of targeted molecules to target the specific molecules associated with the cancer. This kind of treatment that targets diseased cells possesses sweeping “permanent” effects on tumors, in comparison with those three traditional therapies, surgery, radiotherapy and chemotherapy. However, the causes of tumors are various, so the treatment strategy should be designed from different aspects. The targeted therapy is the new technology applied in the current treatments of tumors and can eliminate tumors by inhibiting tumorigenesis and tumor development through various mechanisms.

So far, it has not ever been reported in either China or other countries around the world that the microsphere made from sorafenib and sodium alginate can be applied to the treatment of liver cancer, renal cancer, non-small cell lung cancer, gastric cancer, ovarian cancer, prostate cancer, head and neck tumor, melanoma and other solid tumors with local vascular embolism in target region.

Thus, how to maximize sorafenib's effects on treating tumors has become an urgent technical problem of the field to be resolved.

### DISCLOSURE OF THE INVENTION

One objective of the present invention is to provide a targeted sustained-release sodium alginate microsphere vascular embolizing agent containing sorafenib.

The above-mentioned objective can be achieved via the technical solution described below:

A targeted sustained-release sodium alginate microsphere vascular embolizing agent containing sorafenib, comprising natural carrier sodium alginate and anti-tumor drug sorafenib, where the sorafenib is encapsulated by sodium alginate and the weight ratio of the sorafenib to the sodium alginate is 1:1˜1:30.

Another objective of the present invention is to provide a method for preparing the targeted sustained-release sodium alginate microsphere vascular embolizing agent containing sorafenib.

The above-mentioned objective can be achieved via the technical solution described below:

A preparation method of targeted sustained-release sodium alginate microsphere vascular embolizing agent containing sorafenib, the steps of which are listed below:

(1) Preparation of carrier solution

Sodium alginate is dissolved in physiological saline or water for injection proportionally to prepare a 1 wt %˜7 wt % sodium alginate carrier solution.

(2) Preparation of solidifying solution

Calcium lactate or calcium chloride is weighted proportionally and dissolved in physiological saline or water for injection to obtain a 1 wt %-10 wt % calcium lactate or calcium chloride solution.

(3) Preparation of drug solution

Sorafenib is weighted proportionally and then dissolved in polyethylene glycerol 400 or dimethyl sulfoxide (DMSO) to obtain a sorafenib drug solution.

(4) Preparation of mixture of carrier solution and drug solution

The sorafenib drug solution of Step (3) is mixed with the sodium alginate carrier solution of Step (1) by a high-speed mixer to obtain a mixture solution.

(5) Preparation of targeted sustained-release sodium alginate microsphere containing sorafenib

The mixture solution obtained in Step (4) is reacted with the solidifying solution of Step (2) via a high-voltage electrostatic multihead microsphere generating device to obtain microspheres (or micro gel beads).

A preferred technical solution, characterized in that the high-voltage electrostatic multihead microsphere generating device in Step (5) comprises high-voltage generator, multiplepoint electrode, micro-infusion pump, syringe, tailor-made needle, lifting platform, and sterile glass collector.

A preferred technical solution, wherein the preparation procedure of Step (5) is described below:

1) A 10˜60 ml syringe is fitted with a tailor-made needle and then 10˜60 ml of the mixture solution obtained in Step (4) is aspirated into the syringe;
2) The syringe of Step 1) is fixed in the syringe pushing slot of the micro-infusion pump;
3) The positive interface of the high-voltage electrostatic generator is connected to the tailor-made needles of 2˜12 syringes via multiplepoint electrode; while the negative interface of the high-voltage electrostatic generator is connected, via multiplepoint electrode, to the extensions of 2˜12 b-shape stainless steel rings soaking in the solidifying solution of Step 2); the tailor-made needles are hung above the sterile glass collector which is placed on the lifting platform; the distance between the tip of the tailor-made needle and the surface of liquid in the sterile glass collector is adjusted to 5-20 cm; and once pressing start buttons on the high-voltage electrostatic generator and the micro-infusion pump, the sodium alginate mixture solution containing sorafenib is dropped into the solidifying solution in the sterile glass collector to obtain microspheres (or micro gel beads) called wet beads.

A preferred technical solution, characterized in that the tailor-made needle is made from stainless steel with blunt end.

A preferred technical solution, characterized in that the obtained microspheres (or micro gel beads) are subjected to centrifuge washing or precipitation washing and then stored in a preserving solution to obtain the sodium alginate microspheres (or micro gel beads) containing sorafenib; and the microspheres retain intact without sorafenib leaking during storage.

A preferred technical solution, characterized in that the preserving solution is prepared as described below:

Calcium chloride or calcium lactate is weighted proportionally and dissolved in water for injection to prepare a 3 wt %-15 wt % preserving solution.

A preferred technical solution, characterized in that the particle size range of the microspheres (or micro gel beads) stored in the preserving solution is 50˜100 μm, 70˜150 μm, 100˜200 μm, 100˜300 μm, 150˜450 μm, 300˜500 μm, 500˜700 μm, or 700˜900 μm.

A preferred technical solution, characterized in that the obtained microspheres or micro gel beads are dried via freeze drying (or oven drying) to obtained dry beads, whose particle size range is 20˜60 μm, 30˜75 μm, 50˜100 μm, 70˜150 μm, 80˜250 μm, 150˜300 μm, 250˜500 μm or 500˜700 μm.

A further objective of the present invention is to provide the use of the targeted sustained-release sodium alginate microsphere vascular embolizing agent containing sorafenib.

The above-mentioned objective can be achieved via the technical solution described below:

The use of the targeted sustained-release sodium alginate microsphere vascular embolizing agent containing sorafenib for the manufacturing of medicament for the treatment of liver cancer, lung cancer, ovarian cancer, prostate cancer, head and neck tumor, melanoma and other solid tumors.

The application procedure is described below:

A catheter is inserted into the artery supplying the target organ via interventional radiolography or interventional ultrasonography and then arteriography is performed. The above-mentioned targeted sustained-release sodium alginate microsphere vascular embolizing agent containing sorafenib is chosen according to the arteriogram. When superselecting embolism, microcatheter is preferred and should be manipulated aseptically. The preserving solution in the bottle of the sodium alginate microspheres (wet beads) containing sorafenib is discarded by using syringe. The microspheres are washed with the same amount of physiological saline for 3 times, or are firstly transferred to a sterile bowl from the bottle and then washed with 50˜100 ml physiological saline for 1˜3 times. After discarding the washing fluid, an appropriate amount of contrast agent or the diluted contrast agent is added and mixed with the microspheres to make the microspheres fully suspend in the contrast medium, which is then injected into the focus slowly, in accordance with specific conditions, through the catheter under fluoroscopic control. When the flow of the contrast medium slows down apparently, the embolization is completed. Arteriography is performed once again to evaluate the effectiveness of embolization.

If dry beads are applied, the mircrospheres cannot be used until turning back into wet beads by being soaked in physiological saline half an hour in advance of application.

### Beneficial Effects

By altering the dosage form as well as changing the route of administration, the targeted sustained-release sodium alginate microsphere vascular embolizing agent containing sorafenib of the present invention enables the targeted drugs to be directed to the target region and then to have a rapid, long and focused effect on the cancer tissue. So its advantages lie in good targets, excellent therapeutic effects, negligible harm to normal tissues while killing cancer cells, low toxicity, small amount of required drugs and low treating cost.

The targeted sustained-release sodium alginate microsphere vascular embolizing agent containing sorafenib enhances therapeutic effects by utilizing new techniques to facilitate sorafenib to reach the target region rapidly, to be released sustainably and focused around cancer cells, which reduces the cycle of the drugs, required doses, damage of normal cells and toxicity.

Currently, there are some problems associated with oral administration of sorafenib, including low bioavailability, large doses required, and high toxicity, all of which cause that the medical cost is too high to be acceptable to either doctors or patients. The combination of anti-cancer drug and embolizing agent results in a combined effect when positioning the target region; while the separate normal administrations of these two drugs at the same time have no such an effect. The microsphere encapsulating the targeted drug sorafenib and the arterial vascular embolizing agent allows the concentration of drugs to be maintained in the local tissues for a longer time. The targeted sustained-release sodium alginate microsphere vascular embolizing agent containing sorafenib results in a focused effect by avoiding the first-pass effect whereby the drug is damaged and excreted via systemic circulation and in liver, kidney and other organs, reducing the probability of failure that drug binds to plasma proteins, prolonging the functioning time of the drug, all of which may conquer the defects of oral administration, intravenous chemotherapy and simple drug infusion, including is short retention time in tumor tissues, fast clearance from tumors and inadequate exposure of drug to tumor cells. The clinical pharmacokinetics studies suggests that the quantity of killed cancer cells is increased by 10 to 100 times and the therapeutic effect is doubled when the concentration of anti-cancer drug is doubled within a certain range in the local tissue. Since the targeted sustained-release sodium alginate microsphere vascular embolizing agent containing sorafenib has been developed successfully, the traditional routes of drug administration will be changed and patients would therefore enjoy efficiency, comfort and convenience which are brought by the new-type agent. It will also play an indispensable role in treating solid tumors.

The inventors of the present invention found that the 2˜12 micro-infusion devices in the high-voltage electrostatic multihead microsphere generating device allows more uniform microspheres to be prepared, the yield to be increased, and the microspheres of difference particle size to be produced at the same time.

Hereinafter, the present invention will be further described in the following embodiments. However, these embodiments are not intended to restrict the scope of the present invention.

### DETAILED DESCRIPTION OF THE PREFERRED EMBODIMENTS

### Example 1

### 1. Preparation Before Encapsulating

Treatment of glass wares: The clean glass wares were dried out in the air and then baked in high-temperature oven at 260° C. for 3 hours to kill bacteria and remove pyrogens.

### 2. Preparation of Reagents

(1) Preparation of anti-tumor drug sorafenib solution

10 mg of commercial sorafenib was weighted and added into the above-mentioned glass ware. An appropriate amount of polyethylene glycerol 400 was then dropped till the sorafenib was fully dissolved to obtain 20 ml of sorafenib solution.

(2) Preparation of sodium alginate solution

3 L of 2 wt % sodium alginate solution was prepared by adding physiological saline into sodium alginate while stirring till sodium alginate was fully dissolved.

(3) Preparation of solidifying solution

Adequate calcium chloride was weighted and dissolved in physiological saline to prepare a 3 wt % calcium chloride solution.

(4) Preparation of preserving solution

Adequate calcium chloride was weighted and dissolved in water for injection to prepare a 3 wt % calcium chloride solution, i.e. the preserving solution.

(5) 20 ml of the above-mentioned sorafenib drug solution was mixed with 3 L of the alginate solution by a high-speed mixer to obtain the mixture solution containing sodium alginate and sorafenib.

### 3. Preparation of Sodium Alginate Microsphere Containing Sorafenib

(1) Two 10 ml syringes were fitted with tailor-made needles, respectively, and then the mixture of sorafenib solution and sodium alginate solution was aspirated into the syringes in at least 151 times, respectively.

(2) The syringes mentioned in Step (1) of the preparing process of microsphere were fixed in the syringe pushing slot of the micro-infusion pump.

(3) The positive interface of the high-voltage electrostatic generator was connected to the tailor-made needles of the 2 syringes via multiplepoint electrode; while the negative interface of the high-voltage electrostatic generator was connected, via multiplepoint electrode, to the extensions of the two b-shape stainless steel rings soaking in the solidifying solution of Step (2); the tailor-made needles were hung above the sterile glass collector which was placed on the lifting platform; the distance between the tip of the tailor-made needles and the surface of liquid in the sterile glass collector was adjusted to 12 cm; and once pressing the start buttons of the high-voltage electrostatic generator and the micro-infusion pump, the sodium alginate mixture solution containing sorafenib was dropped into the solidifying solution in the sterile glass collector to obtain microspheres (or micro gel beads) called wet beads. The tailor-made needle was made from stainless steel with blunt end.

(4) Washing: the obtained microspheres (or micro gel beads) were subjected to centrifuge washing or precipitation washing and then stored in the 3 wt % preserving solution. During storage, the microspheres retain intact without sorafenib leaking.

(5) The particle size of the microspheres (or micro gel beads) stored in the preserving solution ranged from 70 to 150 μm.

(6) The obtained sodium alginate microspheres or micro gel beads containing sorafenib were dried out via freeze drying to obtain dry beads, whose particle size ranged from 30 to 75 μm.

The mircrospheres can be used right after turning back into wet beads by being soaked in physiological saline half an hour in advance of application.

### 4. Application to Treating Patients Via Targeted Vessel Embolisation

For the patient suffering from liver cancer, a catheter was inserted into the artery supplying the target organ via interventional radiolography or interventional ultrasonography and then arteriography was performed. The above-mentioned targeted sustained-release sodium alginate microsphere vascular embolizing agent containing sorafenib is chosen according to the arteriogram. When superselecting embolism, microcatheter would be preferred and should be manipulated aseptically. The calcium chloride solution in the bottle of the sodium alginate microspheres (wet beads) containing sorafenib was discarded by using syringe. The microspheres were washed with the same amount of physiological saline for 3 times, or were firstly transferred to a sterile bowl from the bottle and then washed with 50˜100 ml physiological saline for 1˜3 times. After discarding the washing fluid, an appropriate amount of contrast agent or diluted contrast agent was added and mixed with the microspheres to make the microspheres fully suspend in the contrast medium, which was then injected into the focus slowly, in accordance with specific conditions, through the catheter under fluoroscopic control. When the flow of the contrast medium slowed down apparently, the embolization was completed. Arteriography was performed once again to evaluate the effectiveness of embolization.

### Example 2

### 1. Preparation Before Encapsulating

Treatment of glass wares: The clean glass wares were dried out in the air and then baked in high-temperature oven at 260° C. for 3 hours to kill bacteria and remove pyrogens.

### 2. Preparation of Reagents

(1) Preparation of anti-tumor drug sorafenib solution

0.62 g of commercial sorafenib was weighted and added into the above-mentioned glass ware. An appropriate amount of dimethyl sulfoxide (DMSO) was then dropped till the sorafenib was fully dissolved to obtain 500 ml of sorafenib solution.

(2) Preparation of sodium alginate solution 45 L of 1 wt % sodium alginate solution was prepared by adding physiological saline into sodium alginate while stirring till sodium alginate was fully dissolved.

(3) Preparation of solidifying solution

Adequate calcium lactate was weighted and dissolved in physiological saline to prepare a 1 wt % calcium lactate solution.

(4) Preparation of preserving solution

Adequate calcium chloride was weighted and dissolved in water for injection to prepare a 8 wt % calcium chloride solution, i.e. the preserving solution.

(5) 500 ml of the above-mentioned sorafenib solution was mixed with 45 L of the sodium alginate solution by a high-speed mixer to obtain the mixture solution containing sodium alginate and sorafenib.

### 3. Preparation of Sodium Alginate Microsphere Containing Sorafenib

(1) Twelve 60 ml syringes were fitted with tailor-made needles and then the mixture of sorafenib solution and sodium alginate solution was aspirated into the syringes in at least 63 times.

(2) The syringes mentioned in Step (1) of the preparing process of microsphere were fixed in the syringe pushing slot of the microinfusion pump. The parameters of the pump were also adjusted.

(3) The positive interface of the high-voltage electrostatic generator was connected to the tailor-made needles of the 12 syringes via multiplepoint electrode; while the negative interface of the high-voltage electrostatic generator was connected, via multiplepoint electrode, to the extensions of the 12 b-shape stainless steel rings soaking in the solidifying solution of Step (2); the tailor-made needles were hung above the sterile glass collector which was placed on the lifting platform; the distance between the tip of the tailor-made needles and the surface of liquid in the sterile glass collector was adjusted to 5 cm; and once pressing the start buttons of the high-voltage electrostatic generator and the micro-infusion pump, the sodium alginate mixture solution containing sorafenib was dropped into the solidifying solution in the sterile glass collector to obtain microspheres (or micro gel beads) called wet beads. The tailor-made needle was made from stainless steel with blunt end.

(4) Washing: the obtained microspheres (or micro gel beads) were subjected to centrifuge washing or precipitation washing and then stored in the 8 wt % preserving solution. During storage, the microspheres retain intact without sorafenib leaking.

(5) The particle size of the microspheres (or micro gel beads) stored in the preserving solution ranged from 300 to 500 μm.

(6) The obtained sodium alginate microspheres (or micro gel beads) containing sorafenib were dried out via freeze drying (or oven drying) to obtain dry beads, whose particle size ranged from 150 to 300 μm.

The mircrospheres can be used right after turning back into wet beads by being soaked in physiological saline half an hour in advance of application.

### 4. Application to Treating Patients Via Targeted Vessel Embolisation

For the patient suffering from renal cancer, a catheter was inserted into the artery supplying the target organ via interventional radiolography or interventional ultrasonography and then arteriography was performed. The above-mentioned targeted sustained-release sodium alginate microsphere vascular embolizing agent containing sorafenib is chosen according to the arteriogram. When superselecting embolism, microcatheter would be preferred and should be manipulated aseptically. The calcium chloride solution in the bottle of the sodium alginate microspheres (wet beads) containing sorafenib was discarded by using syringe. The microspheres were washed with the same amount of physiological saline for 3 times, or were firstly transferred to a sterile bowl from the bottle and then washed with 50˜100 ml physiological saline for 1˜3 times. After discarding the washing fluid, an appropriate amount of contrast agent or diluted contrast is agent was added and mixed with the microspheres to make the microspheres fully suspend in the contrast medium, which was then injected into the focus slowly, in accordance with specific conditions, through the catheter under fluoroscopic control. When the flow of the contrast medium slowed down apparently, the embolization was completed. Arteriography was performed once again to evaluate the effectiveness of embolization.

### Example 3

### 1. Preparation Before Encapsulating

Treatment of glass wares: The clean glass wares were dried out in the air and then baked in high-temperature oven at 260° C. for 3 hours to kill bacteria and remove pyrogens.

### 2. Preparation of Reagents

(1) Preparation of anti-tumor drug sorafenib solution

6.9 mg of commercial sorafenib was weighted and added into the above-mentioned glass ware. An appropriate amount of dimethyl sulfoxide (DMSO) was then dropped till the sorafenib was fully dissolved to obtain 30 ml of sorafenib solution.

(2) Preparation of sodium alginate solution

2,000 ml of 7 wt % sodium alginate solution was prepared by adding physiological saline into sodium alginate while stirring till sodium alginate was fully dissolved.

(3) Preparation of solidifying solution

Adequate calcium lactate was weighted and dissolved in water for injection to prepare a 10 wt % calcium lactate solution.

(4) Preparation of preserving solution

Adequate calcium lactate was weighted and dissolved in water for injection to prepare a 15 wt % preserving solution.

(5) 30 ml of the above-mentioned sorafenib solution was mixed with 2,000 ml of the sodium alginate solution by a high-speed mixer to obtain the mixture solution containing sodium alginate and sorafenib.

### 3. Preparation of Sodium Alginate Microsphere Containing Sorafenib

(1) Ten 50 ml syringes were fitted with tailor-made needles and then the mixture of sorafenib solution and sodium alginate solution was aspirated into the syringe in at least 4 times.

(2) The syringes mentioned in Step (1) of the preparing process of microsphere were fixed in the syringe pushing slot of the microinfusion pump.

(3) The positive interface of the high-voltage electrostatic generator was connected to the tailor-made needles of the 10 syringes via multiplepoint electrode; while the negative interface of the high-voltage electrostatic generator was connected, via multiplepoint electrode, to the extensions of the 10 b-shape stainless steel rings soaking in the solidifying solution of Step (2); the tailor-made needles were hung above the sterile glass collector which was placed on the lifting platform; the distance between the tip of the tailor-made needles and the surface of liquid in the sterile glass collector was adjusted to 5 cm; and once pressing the start buttons of high-voltage electrostatic generator and the micro-infusion pump, the sodium alginate mixture solution containing sorafenib was dropped into the solidifying solution in the sterile glass collector to obtain microspheres (or micro gel beads) called wet beads. The tailor-made needle was made from stainless steel with blunt end.

(4) Washing: the obtained microspheres (or micro gel beads) were subjected to centrifuge washing or precipitation washing and then stored in the 15 wt % preserving solution. During storage, the microspheres retain intact without sorafenib leaking.

(5) The particle size of the microspheres (or micro gel beads) stored in the preserving solution ranged from 500 to 700 μm.

(6) The obtained sodium alginate microspheres (or micro gel beads) containing sorafenib were dried out via oven drying to obtain dry beads, whose particle size ranged from 250 to 500 μm.

The mircrospheres can be used right after turning back into wet beads by being soaked in physiological saline half an hour in advance of application.

### 4. Application to Treating Patients Via Targeted Vessel Embolisation

For the patient suffering from lung cancer, a catheter was inserted into the artery supplying the target organ via interventional radiolography or interventional ultrasonography and then arteriography was performed. The above-mentioned is targeted sustained-release sodium alginate microsphere vascular embolizing agent containing sorafenib is chosen according to the arteriogram. When superselecting embolism, microcatheter would be preferred and should be manipulated aseptically. The calcium chloride solution in the bottle of the sodium alginate microspheres (wet beads) containing sorafenib was discarded by using syringe. The microspheres were washed with the same amount of physiological saline for 3 times, or were firstly transferred to a sterile bowl from the bottle and then washed with 50˜100 ml physiological saline for 1˜3 times. After discarding the washing fluid, an appropriate amount of contrast agent or diluted contrast agent were added and mixed with the microspheres to make the microspheres fully suspend in the contrast medium, which was then injected into the focus slowly, in accordance with specific conditions, through the catheter under fluoroscopic control. When the flow of the contrast medium slowed down apparently, the embolization was completed. Arteriography was performed once again to evaluate the effectiveness of embolization.

1. A targeted sustained-release sodium alginate microsphere vascular embolizing agent containing sorafenib comprising natural carrier sodium alginate and anti-tumor drug sorafenib, wherein the sorafenib is encapsulated by the sodium alginate and the weight ratio of the sorafenib to the sodium alginate is 1:1˜1:30.

2. A preparation method of the targeted sustained-release sodium alginate microsphere vascular embolizing agent containing sorafenib according to claim 1, wherein the preparation method comprises steps as below:
(1) Preparation of carrier solution
Sodium alginate is dissolved in physiological saline or water for injection proportionally to prepare a 1 wt %˜7 wt % solution then a sodium alginate carrier solution is obtained;
(2) Preparation of solidifying solution
Calcium lactate or calcium chloride is weighted and dissolved in physiological saline or water for injection proportionally to prepare a 1 wt %-10 wt % calcium lactate or calcium chloride solution;
(3) Preparation of drug solution
Sorafenib is weighted proportionally and then dissolved in polyethylene glycerol 400 or dimethyl sulfoxide to obtain a sorafenib drug solution;
(4) Preparation of the mixture of carrier solution and drug solution
The sorafenib drug solution obtained in Step (3) is mixed with the sodium alginate carrier solution obtained in Step (1) by a high-speed mixer to obtain a mixture solution;
(5) Preparation of targeted sustained-release sodium alginate microsphere containing sorafenib
The mixture solution obtained in Step (4) is reacted with the solidifying solution obtained in Step (2) via a high-voltage electrostatic multihead microsphere generating device to obtain microspheres (or micro gel beads).

3. The preparation method of the targeted sustained-release sodium alginate microsphere vascular embolizing agent containing sorafenib according to claim 2, characterized in that the high-voltage electrostatic multihead microsphere generating device in Step (5) comprises high-voltage generator, multiplepoint electrode, micro-infusion pump, syringe, tailor-made needle, lifting platform, and sterile glass collector.

4. The preparation method of the targeted sustained-release sodium alginate microsphere vascular embolizing agent containing sorafenib according to claim 3, characterized in that the preparation procedure of Step (5) is described below:
1) A 10˜60 ml syringe is fitted with a tailor-made needle and then 10˜60 ml of the mixture solution obtained in Step (4) is aspirated into the syringe;
2) The syringe in Step 1) is fixed in the syringe pushing slot of the micro-infusion pump;
3) The positive interface of the high-voltage electrostatic generator is connected to the tailor-made needles of 2˜12 syringes via multiplepoint electrode; while the is negative interface of high-voltage generator is connected, via multiplepoint electrode, to the extensions of 2˜12 b-shape stainless steel rings soaking in the solidifying solution obtained in Step (2); the tailor-made needles are hung above the sterile glass collector which is placed on the lifting platform; the distance between the tip of the tailor-made needle and the surface of liquid in the sterile glass collector is adjusted to 5-20 cm; and once pressing the start buttons of the high-voltage electrostatic generator and the micro-infusion pump, the sodium alginate mixture solution containing sorafenib is dropped into the solidifying solution in the sterile glass collector to obtain the microspheres (or micro gel beads) called wet beads.

5. The preparation method of the targeted sustained-release sodium alginate microsphere vascular embolizing agent containing sorafenib according to claim 4, characterized in that the tailor-made needle is made from stainless steel with blunt end.

6. The preparation method of the targeted sustained-release sodium alginate microsphere vascular embolizing agent containing sorafenib according to claim 5, characterized in that the obtained microspheres or micro gel beads are subjected to centrifuge washing or precipitation washing and then stored in a preserving solution to obtain the sodium alginate microspheres or micro gel beads containing sorafenib; and the microspheres retain intact without sorafenib leaking during storage.

7. The preparation method of the targeted sustained-release sodium alginate microsphere vascular embolizing agent containing sorafenib according to claim 6, characterized in that the preserving solution is prepared as below:
calcium chloride or calcium lactate is weighted proportionally and dissolved in water for injection to prepare a 3 wt %-15 wt % preserving solution.

8. The preparation method of the targeted sustained-release sodium alginate microsphere vascular embolizing agent containing sorafenib according to claim 7, characterized in that the particle size range of the microspheres or micro gel beads stored in the preserving solution is 50˜100 μm, 70˜150 μm, 100˜200 μm, 100˜300 μm, 150˜450 μm, 300˜500 μm, 500˜700 μm, or 700˜900 μm.

9. The preparation method of the targeted sustained-release sodium alginate microsphere vascular embolizing agent containing sorafenib according to claim 8, characterized in that the obtained microspheres or micro gel beads are dried via freeze drying or oven drying to obtain dry beads, whose particle size range is 20˜60 μm, 30˜75 μm, 50˜100 μm, 70˜150 μm, 80˜250 μm, 150˜300 μm, 250˜500 μm or 500˜700 μm.

10. The use of the targeted sustained-release sodium alginate microsphere vascular embolizing agent containing sorafenib according to claim 1 for the manufacturing of medicament for the treatment of solid tumors including liver cancer, lung cancer, renal cancer, ovarian cancer, prostate cancer, and head and neck tumor.
